(19) United States
(12) Patent Application Publication
(10) Pub. No.: US 2022/0195368 A1
(43) Pub. Date: Jun. 23, 2022

(54) FLUID CONDUIT WITH RADIAL EXPANSION OF FLUID FLOW

(71) Applicant: Upside Foods, Inc., Berkeley, CA (US)

(72) Inventor: Nicholas J. GENOVESE, Hayward, CA (US)

(21) Appl. No.: 17/129,662

(22) Filed: Dec. 21, 2020

(51) Int. Cl. C12M 1/00; B01D 69/02; B01D 63/08; B01D 69/06

(52) U.S. Cl. CPC C12M 29/06; B01D 69/02; B01D 63/084; B01D 2325/022; B01D 2325/027; B01D 2313/086; B01D 69/06

(57) ABSTRACT

Disclosed herein are systems, devices, and methods for flowing fluid for radially expanded particle distribution within a laminar flow. In some variations, a system for cultivating tissue may comprise a bioreactor comprising an inlet, a substrate arranged in the bioreactor, and a diffusion module configured to transfer fluid from the inlet to the substrate. The diffusion module may comprise a porous material having at least one tortuous conduit extending between a first surface of the porous material and a second surface of the porous material.

### BACKGROUND

Culturing processes using bioreactors have been developed to mass-produce cells and tissues for various industrial applications including ex-vivo cultivation of tissues for dietary consumption. Most adherent cells of animal origin require attachment to a substrate for growth and may not be maintained suspended in a bioreactor as single cells such as in the case of bacteria or yeast. As a result, the development of efficient biological manufacturing processes that ensure consistent laminar flow, hydrodynamic shear force, and distribution of cells seeded across the entire surface of the cultivation substrate during cultivation processes is desirable.

### SUMMARY

Described herein are systems, devices and methods for expanding fluid flow in a fluid conduit. In some variations, a system for cultivating tissue may comprise a bioreactor comprising an inlet, a substrate arranged in the bioreactor, and a diffusion module configured to transfer fluid from the inlet to the substrate. The diffusion module may comprise a porous material having at least one tortuous conduit extending between a first surface of the porous material and a second surface of the porous material.

In some variations, the at least one tortuous conduit may have a mean pore size of between about 5 nm and about 10 nm, between about 10 nm and about 20 nm, between about 20 nm and about 40 nm, between about 40 nm and about 80 nm, between about 80 nm and about 160 nm, between about 160 nm and about 320 nm, between about 320 nm and about 640 nm, between about 0.64 nm and about 1.2 μm, between about 1.2 nm and about 2.4 nm, between about 2.4 nm and about 4.8 μm, between about 4.8 nm and about 10 nm, between about 10 nm and about 20 nm, between about 20 nm and about 40 nm, between about 40 nm and about 80 nm, between about 80 nm and about 160 nm, between about 160 nm and about 320 μm, between about 320 nm and about 600 nm, or between about 0.6000 mm and about 1.2 mm.

In some variations, the at least one tortuous conduit may have an average arc to chord length ratio of between about 1.1 to about 1.2, about 1.2 to about 1.4, about 1.4 to about 1.6, about 1.6 to about 1.8, about 1.8 to about 2.0, about 2 to about 3, about 3 to about 4, about 4 to about 5, about 5 to about 6, about 6 to about 7, about 7 to about 8, about 8 to about 9, about 9 to about 10, about 10 to about 12, about 12 to about 14, about 14 to about 16, about 16 to about 18, about 18 to about 20, about 20 to about 25, about 25 to about 30, about 30 to about 35, about 35 to about 40, about 40 to about 45, about 45 to about 50, about 50 to about 60, about 60 to about 70, about 70 to about 80, about 80 to about 90, or about 90 to about 100.

In some variations, a porosity of the porous material may be between about 0.1% to about 0.25%, about 0.25% to about 0.50%, about 0.50% to about 1.0%, about 1.0% to about 2.5%, about 2.5% to about 5.0%, about 5.0% to about 10%, about 10% to about 20%, about 20% to about 30%, about 30% to about 40%, about 40% to about 50%, about 50% to about 60% about 60% to about 70% about 70% to about 80%, about 80% to about 90%, about 90% to about 95%, or about 95% to about 99%.

In some variations, the diffusion module may comprise a fluidic focal point aligned with the inlet. In some variations, the diffusion module may have at least one geometrical characteristic that varies with distance from the fluidic focal point. In some variations, the at least one geometrical characteristic may comprise porosity that increases with distance from the fluidic focal point. In some variations, the diffusion module may comprise a planar surface. In some variations, the diffusion module may comprise a concave surface. In some variations, the diffusion module may comprise a convex surface. In some variations, the diffusion module may comprise an open surface in fluidic communication with the inlet.

In some variations, the diffusion module may be one of a plurality of diffusion modules each comprising a fluidic channel comprising the porous material. The system may further comprise a fluidic circuit comprising a manifold in fluidic communication with the inlet and the fluidic channels. In some variations, at least a portion of the fluidic channels may be arranged in parallel. In some variations, at least a portion of the fluidic channels may extend radially from a fluidic focal point of the diffusion module. In some variations, at least a portion of the fluidic channels may be arranged in concentric circuits around a fluidic focal point of the diffusion module. In some variations, the diffusion module may comprise a non-porous material arranged adjacent the porous material. In some variations, the non-porous material may be inlaid in the porous material. In some variations, the surface area ratio of non-porous material to porous material may decrease with distance from a fluidic focal point of the diffusion module.

In some variations, the porous material may comprise at least one material selected from the group consisting of: a silicate, a ceramic, a carbon allotrope, a metal, metallic alloy, a synthetic polymer, a biological polymer, a synthetically-modified biological polymer, a composite, and a resin. In some variations, the device may comprise at least another diffusion module. The at least another diffusion module may be between a second inlet of the bioreactor and the substrate. In some variations, the device may comprise a fluidic control system for controlling introduction of fluid into the inlet of the bioreactor.

In some variations, a method for cultivating tissue may comprise directing a fluid comprising metazoan cells toward a diffusion module. The diffusion module may comprise a porous material having at least one tortuous conduit extending between a first surface of the porous material and a second surface of the porous material, and passing the fluid through the porous material of the diffusion module, thereby radially expanding a laminar flow of the fluid, and seeding the metazoan cells onto a substrate proximate the diffusion module.

In some variations, the substrate may be arranged in a bioreactor. In some variations, the diffusion module may be arranged in the bioreactor between an inlet of the bioreactor and the substrate. In some variations, radially expanding a flow of the fluid may comprise radially distributing the metazoan cells across the diffusion module.

In some variations, directing the fluid toward the diffusion module may comprise directing a first portion of the metazoan cells in a first direction toward the substrate, and directing a second portion of the metazoan cells in second direction toward the substrate. In some variations, the second direction may be opposite the first direction.

In some variations, the porous material may comprise at least one material selected from the group consisting of: a silicate, a ceramic, a carbon allotrope, a metal, a metallic alloy, a synthetic polymer, a biological polymer, a synthetically-modified biological polymer, a composite, and a resin.

In some variations, the diffusion module may comprise an open surface in fluidic communication with the inlet. In some variations, the diffusion module may be one of a plurality of diffusion modules each comprising a fluidic channel comprising the porous material, wherein the system further comprises a fluidic circuit comprising a manifold in fluidic communication with the inlet and the fluidic channels. In some variations, the diffusion module may comprise a non-porous material arranged adjacent to the porous material.

In some variations, the method may comprise culturing the metazoan cells on the substrate. In some variations, culturing the metazoan cells may comprise culturing the metazoan cells into a comestible meat product. In some variations, the fluid may further comprise a cell culture medium.

In some variations, a method may comprise directing a fluid toward a diffusion module, where the diffusion module comprises a porous material having at least one tortuous conduit extending between a first surface of the porous material and a second surface of the porous material, and passing the fluid through the porous material of the diffusion module, thereby radially expanding a laminar flow of the fluid.

In some variations, the fluid may further comprise a liquid comprising cells and wherein radially expanding the laminar flow of the fluid comprises radially distributing the cells across the diffusion module. In some variations, the fluid may comprise a cell culture medium.

In some variations, the method may comprise passing the fluid from the porous material to a substrate to which one or more cells are anchored. In some variations, passing the fluid from the porous material to a substrate surface may reduce variation in hydrodynamic shear force exerted upon the one or more cells anchored to the substrate.

### BRIEF DESCRIPTION OF THE DRAWINGS

The present application may be understood by reference to the following description when taken in conjunction with the accompanying figures.

FIG. 1 shows a schematic of an illustrative variation of a bioreactor including a substrate for seeding of cells under laminar flow.

FIG. 2A shows a schematic view of an illustrative variation of a system for cultivating tissue.

FIGS. 2B-2C shows schematic cross-sectional side views of an illustrative variation of a bioreactor including one or more diffusion modules and a substrate for seeding of cells under laminar flow.

FIG. 3 shows a plan view of an illustrative variation of an open architecture diffusion module.

FIGS. 4A-4E show cross-sectional side views of illustrative variations of open architecture diffusion modules, taken along the line A-A in FIG. 3.

FIGS. 5-8 show radial views of illustrative variations of open architecture diffusion modules comprising non-porous inlays.

FIG. 9 shows a radial cross-sectional side view of an illustrative variation of an open architecture diffusion module.

FIGS. 10-15 show schematic representations of illustrative variations of enclosed architecture diffusion modules.

FIG. 16 is a flow chart of an illustrative variation of a method of using a diffusion module for culturing and harvesting tissue from a substrate.

### DETAILED DESCRIPTION

Described herein are systems and methods for controlling radial expansion of laminar flow in directional flow circuits. Generally, bioreactors developed to culture adherent cells of animal origin, such as for example metazoan cells, often require seeding the cells in a flow regime onto a substrate for cultivation. Where this is the case, the distribution of cells seeded in a fluidic flow path across the cultivation substrate constitutes an important factor for successful cell growth and overall process yield. Where a quantity of cells that otherwise would allow an even distribution of cells across a substrate are stochastically distributed, the presence of cultivation substrate regions with excessive seeding may correspond to inhibition of cell growth by overcrowding and contact inhibition where cells may be deprived of sufficient access to metabolites in the nutrient medium required to support tissue development. Similarly, the presence of cultivation substrate regions with sparse distribution of cells may result in a seeding density of cells below the threshold required to support cell growth and tissue development. Moreover, variations in hydrodynamic shear forces exerted on cells anchored to a cultivation substrate attributable to variation in flow during exposure to nutrient medium may influence biological factors such as cell survival, proliferation, lineage maturation and tissue maturation.

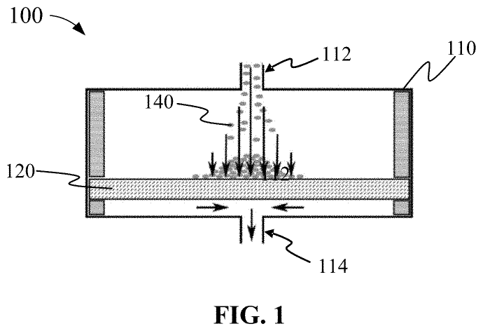

Generally, the systems and methods described herein may find application in various industrial applications including but not limited to food and meat processing, chemical manufacturing, biological machinery, regenerative therapy, textiles, physical augmentation, sewage treatment, cultivation of biofuel feedstocks, microbial carrier and fertilizer distribution in irrigation systems used in landscape, hydroponics, and agriculture, colloidal dispersion of particles within a liquid medium, application of solutes or solutions in aquaria or aquaculture systems, establishing a uniform current in aquatic systems either natural or manmade, processing petrochemicals, and remediation of chemical contamination, food and chemical formulation, food and beverage processing and fermentation, secreted product manufacturing (e.g., milk proteins, egg proteins, immunoglobulin, albumin, peptide growth factors, small molecules), and biological manufacturing of metazoan cell products. FIG. 1 shows a schematic representation of a bioreactor 100 configured to seed cells (e.g., metazoan cells), on a substrate 120. The bioreactor 100 may comprise a housing 110, a substrate 120, and a fluid medium 140 comprising (e.g., inoculated with) cells having individual density (e.g., in g/cm³) that may be slightly higher than that of the surrounding fluid medium 140 (although the individual density may be less in cells comprising fats such as adipocytes). The housing 110 (e.g., enclosure) may include one or more upstream inlets (or upstream conduits) 112, and one or more downstream outlets (or downstream conduits) 114. The upstream inlet 112 may be positioned at any suitable point along the external surface of the housing 110 upstream of substrate 120. For example, as shown in FIG. 1, the upstream inlet 112 may be located at a position on the external surface of the housing 110 that corresponds to the axial center of the housing 110. The upstream inlet 112 may be configured as a path or conduit to flow the fluid medium 140 from upstream components of the bioreactor system 100 to the housing 110, as shown by the downward vertical arrows in FIG. 1. At the upstream inlet 112, the fluid medium 140 transitions to flowing within the housing 110. In some variations, one or more of the inlets 112 and outlets 114 may be disposed along a sidewall of the housing 110. One or more of the inlets 112 and outlets 114 may comprise a predetermined shape and size. For example, the inlets 112 and outlets 114 may be identical, distinct, symmetric, asymmetric, and the like.

The larger mean radius of the housing 110 with respect to the mean radius of the upstream inlet 112 may correspond to an expansion of laminar flow in a radial direction, as the fluid medium 140 passes into the housing 110 from the upstream inlet 112. Said another way, when the fluid medium 140 transitions from the upstream inlet 112 to the housing 110, a diffusion of laminar flow occurs radially from the upstream inlet 112 towards the side walls of the housing 110. In situations where the diffusion of laminar flow in the radial direction does not evenly distribute to housing 110 prior to reaching substrate 120, the fluid medium 140 may experience variations in the hydrodynamic shear force and flow of cells towards the substrate 120, resulting in an uneven fluid flow velocity profile, as well as uneven distribution of single cells seeded on the surface of the substrate 120, as schematically represented in FIG. 1 by the vertical arrows of radially decreasing magnitude pointing toward the downstream outlet 114, and the accumulation of cells near the axial center of the housing 110.

The presence of regions of the substrate 120 with excessive seeding of cells (e.g., uneven distribution of cells across a substrate 120) may correspond to inhibition of cell growth by overcrowding and contact inhibition where cells are deprived of sufficient access to metabolites in the nutrient medium required to support tissue development. Similarly, the presence of regions of the substrate 120 with sparse distribution of cells may result in a seeding density of cells below the threshold required to support cell growth and tissue development. The variations in hydrodynamic shear forces during exposure of seeded cells to a nutrient or cell culture medium may also influence biological factors such as cell survival, proliferation, lineage maturation, and tissue maturation, even in situations where the cells are seeded or anchored to a substrate prior to exposure to the cell culture medium in the bioreactor.

The expansion of laminar flow in the radial direction may be eventually overcome if for example, the housing 110 may be configured as a fluidic conduit path downstream of the upstream inlet 112 to allow sufficient diffusion of laminar flow in the axial direction prior to exposure to the substrate 120. However, the fluidic conduit path length required to achieve adequate radial diffusion of laminar flow may be substantial. In some instances, the extent of fluidic conduit path available downstream of the upstream inlet 112 of the housing 110 may be significantly limited due to other process and/or system requirements such as size maximum size of the bioreactor 100, pressure drop across the bioreactor, and/or heat transfer constraints.

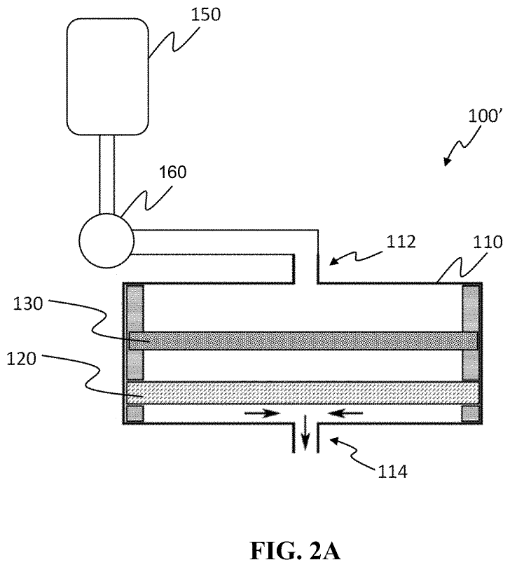

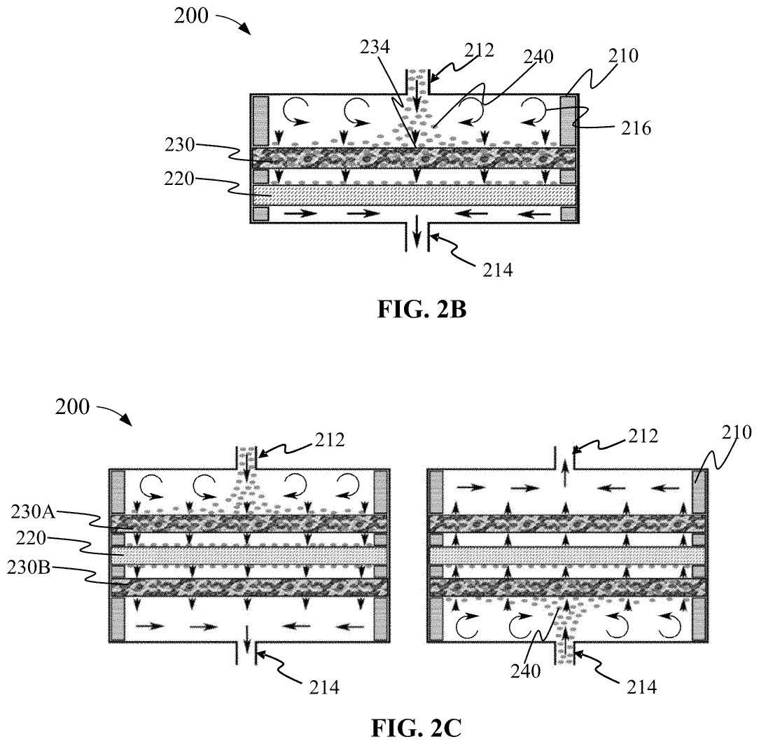

In some variations, the substrate 120 of the bioreactor 100 may be configured to completely cover a cross-sectional area of the housing 120, as for example, when the substrate 120 comprises a cylindrical or disc-shape with a radius similar to the radius of the housing 110. Additionally or alternatively, the substrate 110 may be configured to partially cover the cross-sectional area of the housing 120 and include one or more open sections or regions that facilitate flow from the upstream inlet 112 to the downstream outlet 114, as shown in FIG. 1 by the horizontal arrows located under the substrate 120 and pointing towards the axial center of the housing 110. The portion of the fluid medium 140 that does not contact the substrate 120 as well as the cells not seeded and/or anchored to the surface of the substrate 120 may flow from the housing 110 to the downstream outlet, as shown by the downward arrow located inside the downstream outlet 114 in FIG. 2.

The systems, devices, and methods described herein may accelerate expansion of laminar flow in the radial direction within bioreactors and/or vessels used in biochemical processes, resulting in radial laminar flow expansion and homogeneity. Accelerated radial expansion of laminar flow may, for example, improve radial distribution of laminar flow within the bioreactor, reduce volumetric requirements for flow circuit operation, evenly distribute seeded cells on a surface of cell culture substrate, and homogenize hydrodynamic shear forces to enable predetermined conditions during exposure of seeded cells to nutrient or cell culture medium, resulting in uniform cell survival, and coupled with uniform cell proliferation, lineage maturation, and tissue maturation.

### I. Systems for Cultivating Tissue

Systems such as those described herein for cultivating tissue may carry out biological reactions involving organisms and/or biochemically active substances in a culture media under controlled conditions. Generally, a system for cultivating tissue may comprise a bioreactor with an inlet, a substrate arranged in the bioreactor, and a diffusion module. The diffusion module may be configured to transfer fluid from the inlet to the substrate. The diffusion module may comprise a porous material that has at least one tortuous conduit extending between a first surface of the porous material and a second surface of the porous material. For example, generally, as shown in FIG. 2A, a system 100′ for cultivating tissue may comprise a bioreactor 110 comprising an inlet 112 and an outlet 114. In some variations, the inlet 112 may be in fluidic communication with a fluidic control system 160, and the fluidic control system 160 may further be connected to a vessel 150. The vessel 150 may, for example be used to store a substance appropriate for cultivation of tissue (e.g., cell culture media) as described in further detail below. At least one substrate 120 may be arranged in the bioreactor 110 to support tissue growth, and at least one diffusion module 130 may be configured to transfer fluid from the inlet 112 to the substrate 120 as further described below. Furthermore, the system 100′ may include various additional components and/or subsystems such as an agitation apparatus, a heating and/or cooling apparatus, and one or more sensors and/or probes (not shown).

The bioreactor 110 may include an enclosure (e.g., vessel or chamber) configured to provide a volume suitable to allow for growth (e.g., aseptic growth) of tissue, such as a meat product. The bioreactor 110 may comprise a container configured to hold all the components of the reaction and maintain a set of conditions during reaction. The bioreactor 110 may be composed of one or more materials including glass, quartz, refractory materials, ceramic materials, metals and metal alloys including stainless steel, and/or plastic. The bioreactor 110 may comprise any shape, size or configuration, and may define a cavity comprising any suitable interior volume ranging from tens of milliliters to thousands of liters. The bioreactor 110 may have any suitable shape with predetermined height-to-diameter ratio, such as that suitable to optimize heat and mass transfer within the system. For example, in some instances the bioreactor 110 may comprise a cylindrical shape with a curved base. The interior volume of the bioreactor 110 may be divided between a working volume and a headspace volume. The working volume of the bioreactor 110 may contain a reactive liquid medium, liquid culture medium, gas bubbles, solid substrates as well as suspended animal cell microorganisms. The headspace of the vessel may allow additional volume, typically accessible to gas phase content.

In some variations, the bioreactor 110 may optionally include one or more fluidic ports that may be configured as inlets and/or outlets of components in the reaction. Reaction components may include: (i) oxygen to the culture media (i.e., in the case of aerobic reactions); (ii) exhaust of byproduct gases like CO₂; (iii) feeding culture media and/or feedstock to the reactor; and (iv) drain line of the final products. The bioreactor 110 may be operated in a continuous process or in a batch process. The size, location, and configuration of the fluidic ports may be based on the type of application. In some variations, a port may comprise common ports including a sanitary end, 25 mm safety, rupture discs, flanged motor mounts, manways, combinations thereof, and the like. In order to prevent contamination, the fluidic ports may comprise a seal system configured to maintain a sterile boundary. For example, in some instances port seals may include O-rings and/or gasket type seals.

At least one substrate 120 may be housed within the bioreactor 110. In some variations, the substrates may be configured to mimic the environment enabling growth and propagation of cell cultures. The substrates may vary in size and shape and may be suitable for fitting multiple culture vessels including, but not limited to, bioreactors, wells, petri dishes, plates, flasks, bottles, tanks, boxes and fixed surfaces. The substrates may be composed of materials including, but not limited to, polymers such as polychlorotrifluoroethylene, polyetherimide, polysulfone, polystyrene, polycarbonate, polypropylene, silicone, polyetheretherketone, polymethylmethacrylate, nylon, acrylic, polyvinylchloride, phenolic resin, petroleum-derived polymers, polyethylene, polyterephthalate, metals such as stainless steel, titanium, aluminum, cobalt-chromium, chrome, and alloys, and inorganic materials such as glass, silicates, ceramics and combination or composites thereof. In some instances, the substrates may be membranes configured to contain coatings and/or conjugated peptides for cell adherence and retention. The substrates may include patterned textures including one or more features, shapes, directionalities, dimensions (e.g., length, width, depth, thickness, curvature, volume, area, etc.), densities, periodicities, surface roughness, porosities, and the like. The patterned texture may be composed of linear features that may be continuous, non-continuous, dotted, dashed, repeating, periodic, random, constant width, varying width, and combinations thereof. The substrate patterns may be characterized by a direction and shape using, for example, channels, recesses, ridges, blind holes, hills-and-valleys, undercuts, scratches, edges and combinations thereof.

In some variations, a fluidic control system 160 (e.g., fluid controller) may optionally be configured to control the flow of different gases and/or liquids in and out of the bioreactor 110 (e.g., to modulate physical parameters such as flow rate and pressure of fluid), such as from the vessel 150. The fluidic control system 160 may comprise one or more pumps configured to introduce a fluid to the vessel 150 and/or remove residual fluid out of the vessel 150 after the reaction has finished, compressors to pressurize air and allow fluid flow into the bioreactor 110, valves to open and/or close either manually or automatically different streams, and mass flow controllers to precisely control the amount of gases incorporated into the bioreactor 110. For example, a fluidic control system 160 may include one or more peristaltic pumps comprising a drive (e.g., motor), a pump head and tubing configured to avoid contamination. Some fluidic control systems 160 include compressors that may be configured to operate oil-free to avoid contamination of the media with air or other compressed gases in the bioreactor 110. Some fluidic control systems 160 may include one or more valves such as on-and-off control ball valves, large pipes butterfly valves, one-way flow check valve, sanitary flow diaphragm valve, flow regulation glove valve, and slow pressure release needle valves.

In some variations, the vessel 150 may be used to store any substance appropriate for cultivation of tissues in the bioreactor 110. For example, the vessel may contain fluids, such as cell culture medium, buffers, sterile water, enzyme preparations, or any other liquids applicable in cell culture. In some variations the vessel 150 may be in fluidic communication with the bioreactor 110. In some variations the vessel 150 may contain gases, such as oxygen, carbon dioxide, nitrogen, argon, or any other gas applicable in cell culture. In some variations the vessel 150 may contain cells suspended in cell culture medium. In some variations the vessel 150 may contain contractile stimuli, such as small molecules, calcium channel modulators, or acetylcholine suspended in cell culture medium.

In some variations, the system 100′ may include an agitation apparatus (e.g., agitator) configured to maintain a homogeneous cell culture media with well-distributed components within the vessel 150. Several types of agitation apparatuses may be used including, but not limited to, mechanical agitators or impellers, vessel rocking apparatuses, bubblers, internally mixing media, combinations thereof, and the like. Mechanical agitation apparatuses may comprise a motor connected to a speed reducer, a shaft with shaft supports, and one or more impeller blade configured to move the media inside the vessel 150. For example, the vessel 150 may include one or more of a Rushton turbine, pitched-blade, and marine propeller-like impellers. In some variations, mechanical agitation apparatuses may be placed either top-drive or bottom-drive agitation in the center of the vessel 150 or some degrees offset. In some instances, the impellers of the agitation apparatus may create a different type of flow in the vessel 150 axial and radial or tangential to the walls of the vessel 150. In some variations, the flow pattern in the vessel 150 may be turbulent or laminar. In some instances, internal baffles may be configured to generate a turbulent flow that breaks up laminar flow in the vessel 150r. In some variations, the agitation apparatus may include one or more baffles. The baffles may comprise divisions inside the walls of the vessel 150 that change the flow of the media during mixing. For example, rocker vessels may be equipped with a bag as a container deposited on a moving platform to produce rocking motion of the fluid inside for agitation. Air lift vessels may be configured to use air bubbles to distribute oxygen to the cell culture media and create mixing.

In some variations, a heating and/or cooling apparatus may be configured to control the temperature of the cell culture media. Heating and/or cooling apparatuses used in bioreactor systems may include jacketed reactors, heating coils, and heating blankets. In some instances, small bioreactor systems may use condensers as the heat exchanger system for the reactor.

Additionally or alternatively, in some variations, one or more sensors and/or probes of a bioreactor may be configured to control several characteristics and/or conditions relevant to a biological reaction. Measured and/or monitored conditions may include temperature, concentration of dissolved oxygen (dO), foaming, pH, temperature, mixing, and supplementation of nutrients. In some bioreactor systems, temperature may be monitored using thermocouples, and/or platinum, zinc or copper resistance temperature detectors located inside the bioreactor. In some bioreactors, dissolved oxygen may be measured with a sensor such as a Clark-type sensor, a galvanic sensor, and/or an optical sensor. In some bioreactors, generation of foam may be controlled with a foam control system comprising two probes. For example, a first probe may be immersed in the liquid and a second probe may be arranged in a headspace area (e.g., located at the limit position that the formed foam may reach before addition of antifoam additives). In some bioreactors, pH may be measured with traditional electrochemical glass pH electrodes such as a potentiometer. In other bioreactors, pH may be measured with more flexible pH sensors including optical sensors that may be small and low cost. In some bioreactors, a pH control system may measure in-situ values in the reactor with a probe and adjust the pH by addition of an alkali or acid solution.

FIG. 2B depicts a schematic representation of a bioreactor 200 which may, for example, be included in a system 100′ as described above. In some variations, the bioreactor 200 may be configured to facilitate radial expansion of laminar flow of a fluid medium including particles (e.g., nutrients, cells, seeding cells, metazoan cells) with at least one diffusion module 230. The bioreactor 200 may include a housing 210, a fluid medium 240 comprising one or more of a liquid medium (e.g., liquid conducting medium), particles, and inoculated single cells having an individual density (e.g., in g/cm³) slightly greater than that of the surrounding medium. The bioreactor 200 may further include a substrate 220 and one or more diffusion modules 230. The housing 210 may be any suitable size, shape, and configuration. For example, in some variations, the housing 210 may be cylindrical in shape and have a substantially circular radial cross-sectional shape (e.g., tubular structure). In other variations, the housing 210 may have a spherical, conical, box, or any other suitable shape. The housing 210 may comprise an upstream inlet (or upstream conduit) 212, and a downstream outlet (or downstream conduit) 214. The upstream inlet 212 may be positioned at any suitable point along the external surface of the housing 210. In some variations, the bioreactor 200 may be coupled to one or more pumps. Additionally or alternatively, in some variations, the upstream inlet 212 may be located at a position on the external surface of the housing 210 that is elevated with respect to the ground such that fluids transported within the upstream inlet 212 may flow into the housing 210 based at least on the action of gravitational forces. In other variations, the upstream inlet 212 may be located at a position on the external surface of the housing 210 that is not elevated with respect to the ground, and fluids may be transported into the housing 210 by the aid of one or more pumps.

In some variations, the upstream inlet 212 may be a tubular structure fluidically coupled (e.g., in fluid communication) to the housing 210 and located at a position that corresponds to the axial center of the housing 210. The upstream inlet 212 may be configured as a path or conduit to flow the fluid medium 240 from upstream components of the bioreactor system 200 into the housing 210, as shown by the downward vertical arrows aligned with the axial center of the housing 210 in FIG. 2A. At a distal end of the upstream inlet 212, the fluid medium 240 may transition to flow within the housing 210. As mentioned above with respect to the bioreactor 100, the larger mean radius of the housing 210 with respect to the mean radius of the upstream inlet 212 allows an expansion of laminar flow in the radial direction as the fluid medium 240 exits the upstream inlet 212 and into housing 210. Said a different way, when the fluid medium 240 transitions from an end of the upstream inlet 212 to the housing 210, diffusion of flow may expand in the radial direction originating from the upstream inlet 212 towards the side walls of the housing 210 by a combination of laminar and turbulent flow.

FIG. 2C illustrates an exemplary variation of cell seeding on opposite sides of a substrate 220. For example, flow comprising one or more fluids and particulates (e.g., media and cells) may be directed from upstream inlet 212 into housing 210 as shown in the left side of FIG. 2C, where diffusion of flow may expand in the radial direction originating from inlet 212 toward the sidewalls of the housing 210 by a combination of laminar and turbulent flow 216. Once a first side of the substrate 220 has been seeded, a second side of the substrate 220 opposite the first side of the substrate 220 may be seeded by reversing the direction of fluid flow, as shown in the right side of FIG. 2C. For example, flow comprising one or more fluids and particulates (e.g., media and cells) may be directed from downstream outlet 214 into housing 210 as shown in the left side of FIG. 2C, where diffusion of flow may expand in the radial direction originating from outlet 214 toward the sidewalls of the housing 210 by a combination of laminar and turbulent flow.

In some variations, the ratio between a radius of the upstream inlet 212 to a radius of the housing 210 may be between about 1.1 to about 1.0:1:2, about 1.0:1:2 to about 1.0:1.3, about 1.0:1.3 to about 1.0:1.4, about 1.0:1.4 to about 1.0:1.5, about 1.0:1.5 to about 1.0:1.6, about 1.0:1.6 to about 1.0:1.7, about 1.0:1.7 to about 1.0:1.8, about 1.0:1.8 to about 1.0:1.9, about 1.0:1.9 to about 1:2, about 1:2 to about 1:3, about 1:3 to about 1:4, about 1:4 to about 1:5, about 1:5 to about 1:6, about 1:6 to about 1:7, about 1:7 to about 1:8, about 1:8 to about 1:9, about 1:9 to about 1:10, about 1:10 to about 1:20, about 1:20 to about 1:30, about 1:30 to about 1:40, about 1:40 to about 1:50, about 1:50 to about 1:60, about 1:60 to about 1:70, about 1:70 to about 1:80, about 1:80 to about 1:90, about 1:90 to about 1:100, about 1:100 to about 1:200, about 1:200 to about 1:400, about 1:400 to about 1:500, about 1:500 to about 1:600, about 1:600 to about 1:700, about 1:700 to about 1:800; about 1:800 to about 1:900, about 1:900 to about 1:1,000, including all values and sub-ranges in-between.

In some variations, the fluid medium 240 may be a liquid and/or a gas. In some variations, the fluid may be a Newtonian fluid, a non-Newtonian fluid, a compressible fluid and/or an incompressible fluid. In some variations, the fluid may include particulate matter whose mean diameter is between about 5 nm to about 10 nm, about 10 nm to about 20 nm, about 20 nm to about 40 nm, about 40 nm to about 80 nm, about 80 nm to about 160 nm, about 160 nm to about 320 nm, about 320 nm to about 640 nm, about 0.5 μm to about 1.0 μm, about 1.0 μm to about 2.5 μm, about 2.5 μm to about 5 μm, about 5 μm to about 10 μm, about 10 μm to about 20 μm, about 20 μm to about 40 μm, about 40 μm to about 80 μm, about 80 μm to about 160 μm, about 160 μm to about 320 μm, about 320 μm to about 620 μm, about 620 μm to about 1.5 mm, including all values and sub-ranges in-between.

In some variations, the fluid medium 240 may transition from the upstream inlet 212 to the housing 210 and the diffusion module 230, located between the upstream inlet 212 and the substrate 220. The point on the surface of the diffusion module 230 that intersects the trajectory of the fluid medium 240 upon entering the bioreactor 200 corresponds to a fluidic focal point. The fluidic focal point represents the point where the flow of the fluid 240 tends to be laminar upon first contacting the diffusion module 230. For example, FIG. 2B shows the fluid medium 240 entering the housing 210 with a downward trajectory parallel to the axial axis of the bioreactor 200, and intersecting the diffusion module 230 at fluidic focal point 234, which in this case also corresponds to the center of the diffusion module 230. The fluid medium 240 entering the housing 210 also comprises a turbulent flow 216 component between the inlet 212 and diffusion module 230.

In some variations, exposure of the fluid medium 240 to the diffusion module 230 may correspond to channeled flow of the fluid medium 240 through the diffusion module 230, which may: (1) direct turbulent flow 216 in the radial direction, as shown in FIG. 2B; and (2) transfer radially expanded laminar flow of fluid medium 240 and inoculated cells upon exiting the diffusion module 230 towards the substrate 220. The diffusion module 230 may be configured to accelerate diffusion of laminar flow in the radial direction by providing localized flow paths that may be progressively divided as flow is channeled through the diffusion module 230, thereby re-directing turbulent flow into laminar flow. To provide such localized flow paths, the diffusion module 230 may comprise a porous medium characterized by one or more of porosity, tortuosity, mean pore size, and pore size distribution. As used herein, porosity may refer to the percentage void volume present within the diffusion module 230, as described in more detail herein. Example variations of the diffusion module are also described in further detail below.

In some variations, upon exiting the diffusion module 230, the radially expanded laminar flow of fluid medium 240 may be directed towards the substrate 220 to seed the cells with an even distribution, as shown in FIGS. 2A and 2B. The radially expanded laminar flow may correspond to an evenly distributed hydrodynamic shear force exerted on the cells seeded or anchored to the substrate 220, which may positively influence the consistency of biological factors across the substrate such as cell survival, proliferation, lineage maturation and tissue maturation, even in situations where the cells may be seeded or anchored to a substrate prior to exposure to the cell culture medium in the bioreactor 200.

In some variations, the radial expansion of laminar flow accelerated by the diffusion module 230 upon exposure to fluid medium 240, may be increased by about 5% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 100%, about 100% to 200%, about 200% to about 300%, about 300% to about 400%, about 400% to about 500%, about 500% to about 600%, about 600% to about 700%, about 700% to about 800%, about 800% to about 900%, about 900% to about 1,000%, about 1,000% to about 2,000%, about 2,000% to about 3,000%, about 3,000% to about 4,000%, about 4,000% to about 5,000%, about 5,000% to about 6,000%, about 6,000% to about 7,000%, about 7,000% to about 8,000%, about 8,000% to about 9,000%, about 9,000% to about 10,000%, about 10,000% to about 20,000%, about 20,000% to about 30,000%, about 30,000% to about 40,000%, about 40,000% to about 50,000%, about 50,000% to about 60,000%, about 60,000% to about 70,000%, about 70,000% to about 80,000%, about 80,000% to about 90,000%, about 90,000% to about 100,000%, including all values and sub-ranges in-between.

As discussed herein with respect to bioreactor 200, substrate 220 may be configured to partially cover the cross-sectional area of the housing 210, and may include one or more areas that facilitate flow from the surface of the substrate 220 to the downstream outlet 214, as shown in FIGS. 2B and 2C by the horizontal arrows located under the substrate 220 and pointing towards the axial center of the housing 210. The fluid medium 240 not contacting the substrate 220 and the cells not seeded and/or anchored to the surface of the substrate 220 may flow from the housing 210 to the downstream outlet 214, as shown by the arrow located inside the downstream outlet 214 in FIG. 2B.

In some variations, unidirectional laminar flow may be insufficient or suboptimal for seeding the targeted substrate surfaces based on the geometry of the substrate 220 and the surfaces of the substrate 220 targeted for seeding, particularly where the surfaces are oblique or facing away from the upstream inlet 212. In these circumstances, the bioreactor 200 may include diffusion modules 230 on either side or surface of the substrate 220 (e.g., above and below), as shown in FIG. 2C. In some variations, fluid medium 240 may flow in an alternating manner. For example, fluid medium 240 may first flow from the upstream inlet 212 through a first diffusion module 230A and into the upstream facing side of the substrate 220, as shown in the left side bioreactor of FIG. 2B. After a sufficient exposure to the fluid medium 240 conducive to attachment of single cells to the substrate 220 under laminar flow has taken place, the flow of the bioreactor 200 may be reversed under a second flow. During reversed flow, fluid medium 240 may flow from the downstream outlet 214, through the diffusion module 230B and over the downstream facing side of the substrate 220, as shown in the right side bioreactor of FIG. 2B. The relative angle of the alternating flow trajectories may be about 180 degrees. In some variations, the relative angle of the alternating flow trajectories may be between about 185° to about 175°, about 175° to about 165°, about 165° to about 155°, about 155° to about 145°, about 145° to about 135°, about 135° to about 125°, about 125° to about 115°, about 115° to about 105°, about 105° to about 95°, about 95° to about 85°, about 85° to about 75°, about 75° to about 65°, about 65° to about 55°, about 55° to about 45°, about 45° to about 35°, about 35° to about 25°, about 25° to about 15°, about 15° to about 5°, including all values and sub-ranges in-between.

In some variations, unidirectional laminar flow may be insufficient or suboptimal for seeding substrate surfaces with cells. In such configurations, fluid medium 240 may, for example, flow in an alternating manner including two alternating trajectories. In some variations, the number of alternating trajectories may be between about 1 to about 5, about 5 to about 10, about 10 to about 15, about 15 to about 30, about 30 to about 50, about 50 to about 100, about 100 to about 250, and about 250 to about 500, including all values and sub-ranges in-between.

### Diffusion Module

As described above, the diffusion module may function to help accelerate radial expansion of laminar flow of fluid entering the bioreactor (e.g., to help evenly distribute cells for seeding across the substrate). The diffusion module 230 may include a porous material having at least one tortuous conduit extending between a first surface of the porous material and a second surface of the porous material.

In some variations, the diffusion module 230 and/or portions thereof may be formed or constructed of one or more suitable materials including silicate, ceramic, carbon allotrope, metal, metallic alloy, synthetic polymer, biopolymer, composites, resins, combinations thereof, and the like. In some variations, the diffusion module 230 may be formed or constructed of biocompatible materials. In some variations, the biocompatible materials may be selected based on one or more properties of the constituent material such as, for example, stiffness, toughness, durometer, bioreactivity, etc. Examples of suitable biocompatible materials include metals, silicates, ceramics, or polymers. Examples of suitable metals include stainless steel, titanium, nickel, iron, tin, chromium, copper, and/or alloys thereof. A polymer material may be biodegradable or non-biodegradable. Examples of suitable biodegradable polymers include polylactides, polyglycolides, polylactide-co-glycolides, polyanhydrides, polyorthoesters, polyetheresters, polycaprolactones, polyesteramides, poly(butyric acid), poly(valeric acid), polyurethanes, biodegradable polyamides (nylons), and/or blends and copolymers thereof. Examples of non-biodegradable polymers include non-degradable polyamides (nylons), polyesters, polycarbonates, polyacrylates, polymers of ethylene-vinyl acetates and other acyl substituted cellulose acetates, non-degradable polyurethanes, polystyrenes, polyvinyl chloride, polyvinyl fluoride, poly(vinyl imidazole), chlorosulphonate polyolefins, polyethylene oxide, and/or blends and copolymers thereof.

In some variations, the diffusion module 230 may be any suitable shape, size and configuration. In some variations, the diffusion module 230 may correspond to an open configuration, also referred to as open architecture, in which fluid is received across an open surface (e.g., exposed surface not enclosing the fluid as part of an enclosed architecture such as that described below). For example, in the variation shown in FIG. 2B, the diffusion module 230 may correspond to an open architecture having an exposed, generally planar surface that receives the fluid. In some variations, a diffusion module 230 may correspond to an open architecture having a circular, square, rectangular, triangular, elliptical, trapezoidal and/or any other polygonal shape. In some variations, the diffusion module 230 shape may conform to the shape and/or geometry of a corresponding housing 210 such that flow bypass around the diffusion module 230 is not permitted. In some variations, a diffusion module 230 may correspond to an open architecture having any suitable irregular shape, cross-section, and/or the like.

In some variations, the geometric alignment and/or disposition of a diffusion module within a housing may assume any suitable configuration. As shown in FIG. 2B, an open architecture diffusion module 230 may be a circular or disc-like shape positioned within the housing of the bioreactor 200 such that the center of the disc-like diffusion module 230 and the upstream inlet 212 may be axially aligned within the housing 210 such that the fluidic focal point 234 corresponds to the center of the diffusion module 230. In other variations, the open architecture diffusion module 230 may be disposed within the housing 210 such that the geometrical center of the diffusion module 230 is not aligned with respect to a major axis of the housing 210 and/or the fluidic focal point 234 does not correspond to the center of the module 210. For example, in some variations, the open architecture diffusion module 230 may be positioned within the housing 210 such that the fluidic focal point 234 is located at a point closer to the perimeter of the diffusion module 230.

In some variations, the mean radius of the open architecture diffusion module 230 may have a length of about 0.5 mm to about 1.0 mm, about 1.0 mm to about 2.0 mm, about 2.0 mm to about 5.0 mm, about 5.0 mm to about 10.0 mm, about 1.0 cm to about 2 cm, about 2 cm to about 3 cm, about 3 cm to about 6 cm, about 6 cm to about 10 cm, about 10 cm to about 20 cm, about 20 cm to about 50 cm, about 0.5 m to about 1.0 m, about 1 m to about 2 m, about 2 m to about 5 m, about 5 m to about 10 m, about 10 m to about 20 m, about 20 m to about 30 m, 30 m to about 65 m, including all ranges and sub-values in-between.

In some variations, the open architecture diffusion module 230 may be disposed within the housing 210 such that the trajectory of the fluid medium upon entering the housing 210 enters orthogonally into the diffusion module 230, as shown by the arrows pointing downward on the variation of FIGS. 2B and 2C, thereby transitioning turbulent flow into laminar flow. The diffusion module 230 and/or the upstream inlet 212 may be disposed within the housing 210 such that the trajectory of the fluid medium upon entering the housing 210 intersects the diffusion module at a predetermined angle. In some variations, said predetermined angle may be between about 0 degrees and about 90 degrees, including all ranges and sub-values in-between.

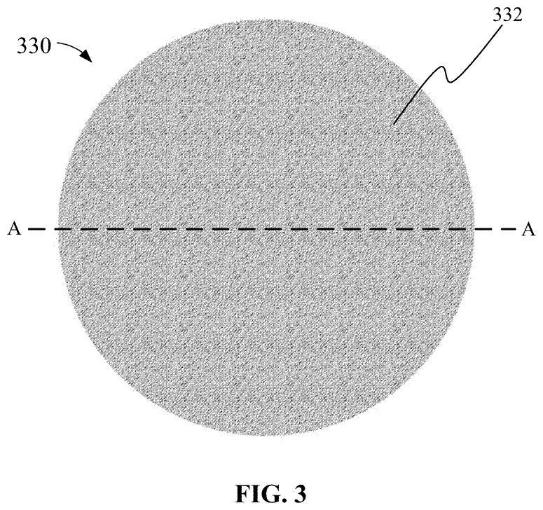

FIG. 3 illustrates an open architecture diffusion module 330 comprising a disc-like shape including a first circular exterior surface 332 located on a first side of the diffusion module 330, and a second circular exterior surface (not shown) located on a second side of the diffusion module 330 (e.g., opposite the first surface 332). The diffusion module 330 may comprise a porous medium characterized by porosity, mean pore size and pore size distribution, where porosity refers to the percent void volume present within the diffusion module 330. The diffusion module 330 shown in FIG. 3 may include networks of interconnected tortuous channels and open pores configured for fluidic permeation and flow (e.g., passage of fluid, fluid flow) from the first exterior surface 332 to the second exterior surface or vice versa, depending on the geometric arrangement and/or configuration of the diffusion module 330 within a housing 310 and/or any available fluidic point of entry, understood as a point from which a flow of fluid medium may originate. The porosity of the diffusion module 330 may define a plurality of paths for fluid flow that initiate at the exterior surfaces of the diffusion module 330, where open pores may be in fluid communication with networks of interconnected tortuous channels disposed within the diffusion module 330 to facilitate distribution of laminar flow in a manner stochastically radial from the exterior surfaces of the diffusion module 330 and/or any available fluidic point of entry. In some variations, a tortuosity of the diffusion module 330 may create non-linear fluid flow paths within the diffusion module 330. Said another way, the tortuosity of the diffusion module 330 may be defined as a relative curvature (e.g., twists and turns) of the flow paths provided by the networks of interconnected channels within the diffusion module.

The tortuosity of the diffusion module 330 and the corresponding tortuosity of the interconnected channels within the diffusion module 330 may be described by an arch ratio of a ratio of the length of the channel to the distance between the channel's ends. For example, in a diffusion module comprising a tortuosity described by an arch ratio of 2.0, the length of the channels is two times greater than the distances between the channel's ends. For diffusion modules that include a plurality of interconnected channels having different or dissimilar lengths and/or distances between the channel's ends, the tortuosity of the diffusion module may be described by considering an average arch ratio. In some variations, the average arch ratio may be an arithmetic average of the arch ratio for each channel within the diffusion module.

In some variations, the diffusion module 330 may have a tortuosity described by an average arc to chord length ratio greater than or equal to about 1.1, greater than or equal to about 1.2, greater than or equal to about 1.4, greater than or equal to about 1.6, greater than or equal to about 1.8, greater than or equal to about 2.0, greater than or equal to about 3, greater than or equal to about 4, greater than or equal to about 5, greater than or equal to about 6, greater than or equal to about 7, greater than or equal to about 8, greater than or equal to about 9, greater than or equal to about 10, greater than or equal to about 12, greater than or equal to about 14, greater than or equal to about 16, greater than or equal to about 18, greater than or equal to about 20, greater than or equal to about 25, greater than or equal to about 30, greater than or equal to about 35, greater than or equal to about 40, greater than or equal to about 45, greater than or equal to about 50, greater than or equal to about 60, greater than or equal to about 70, greater than or equal to about 80, greater than or equal to about 90, and greater than or equal to about 100, including all values and sub-ranges in-between.

In some variations, the diffusion module 330 may have a tortuosity described by an average arc to chord length ratio less than or equal to about 100, less than or equal to about 90, less than or equal to about 80, less than or equal to about 70, less than or equal to about 60, less than or equal to about 50, less than or equal to about 45, less than or equal to about 40, less than or equal to about 35, less than or equal to about 30, less than or equal to about 25, less than or equal to about 20, less than or equal to about 18, less than or equal to about 16, less than or equal to about 14, less than or equal to about 12, less than or equal to about 10, less than or equal to about 9, less than or equal to about 8, less than or equal to about 7, less than or equal to about 6, less than or equal to about 4, less than or equal to about 3, less than or equal to about 2, less than or equal to about 1.8, less than or equal to about 1.6, less than or equal to about 1.4, less than or equal to about 1.2, and less than or equal to about 1.1, including all ranges and sub-values in-between. Combinations of the above-referenced ranges for the tortuosity of the open architecture diffusion module 330 are also possible (e.g., greater than or equal to about 1.0 and less than or equal to about 70, greater than or equal to about 14 and less than or equal to about 100).

As described herein, the open pores and interconnected tortuous channels of the diffusion module 330 may correspond to pathways for fluid flow within the diffusion module. The lower limit on the mean pore size of the open pores and interconnected channels defining the porosity of the diffusion module 330 may be bounded by the size of the cells present in the fluid medium, the proclivity of particulate matter (e.g., cells), to occlude the open pores distributed on the surface of the diffusion module 330, and the ability of the particulate matter to flow within the continuous or catenary interconnected network within the module. In some variations, the mean pore size of the diffusion module 330 may be greater than or equal to about 5 nm, greater than or equal to about 10 nm, greater than or equal to about 20 nm, greater than or equal to about 40 nm, greater than or equal to about 80 nm, greater than or equal to about 160 nm, greater than or equal to about 320 nm, greater than or equal to about 640 nm, greater than or equal to about 1.2 μm, greater than or equal to about 1.2 μm, greater than or equal to about 4.8 μm, greater than or equal to about 10 μm, greater than or equal to about 20 μm, greater than or equal to about 40 μm, greater than or equal to about 80 μm, greater than or equal to about 160 μm, greater than or equal to about 320 μm, greater than or equal to about 600 μm, greater than or equal to 1.2 mm, including all ranges and sub-values in-between.

In some variations, the mean pore size of the diffusion module may be less than or equal to about 1.2 mm, less than or equal to about 620 μm, less than or equal to about 310 μm, less than or equal to about 160 μm, less than or equal to about 80 μm, less than or equal to about 40 μm, less than or equal to about 20 μm, less than or equal to about 10 μm, less than or equal to about 5 μm, less than or equal to about 2.5 μm, less than or equal to about 1.2 μm, less than or equal to about 640 nm, less than or equal to about 320 nm, less than or equal to about 160 nm, less than or equal to about 80 nm, less than or equal to about 40 nm, less than or equal to about 20 nm, or less than or equal to about 10 nm, including all ranges and sub-values in-between.

Combinations of the above-referenced ranges for the mean pore size of the open architecture diffusion module 330 are also possible (e.g., greater than or equal to about 5 nm and less than or equal to about 4.8 μm, greater than or equal to about 160 nm and less than or equal to about 1.2 mm), including all ranges and sub-values in-between.

In some variations the porosity of the diffusion module, where porosity refers to the percentage void volume present within the diffusion module, may be greater than or equal to about 0.1%, greater than or equal to about 0.25%, greater than or equal to about 0.50%, greater than or equal to about 0.5%, greater than or equal to about 1%, greater than or equal to about 2.5%, greater than or equal to about 5%, greater than or equal to about 10%, greater than or equal to about 20%, greater than or equal to about 30%, greater than or equal to about 40%, greater than or equal to about 50%, greater than or equal to about 60%, greater than or equal to about 70%, greater than or equal to about 80%, greater than or equal to about 90%, greater than or equal to about 95%, or greater than or equal to about 99%, including all ranges and sub-values in-between.

In some variations, the porosity of the diffusion module may be less than or equal to about 99%, less than or equal to about 95%, less than or equal to about 90%, less than or equal to about 80%, less than or equal to about 70%, less than or equal to about 60%, less than or equal to about 50%, less than or equal to about 40%, less than or equal to about 30%, less than or equal to about 20%, less than or equal to about 10%, less than or equal to about 5%, less than or equal to about 2.5%, less than or equal to about 1%, less than or equal to about 0.5%, or less than or equal to about 0.25%, including all ranges and sub-values in-between. Combinations of the above-referenced ranges for the porosity of the open architecture diffusion module are also possible (e.g., greater than or equal to about 0.25% and less than or equal to about 99%, greater than or equal to about 5% and less than or equal to about 70%).

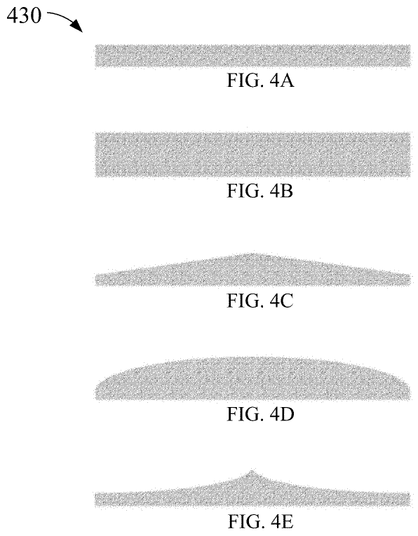

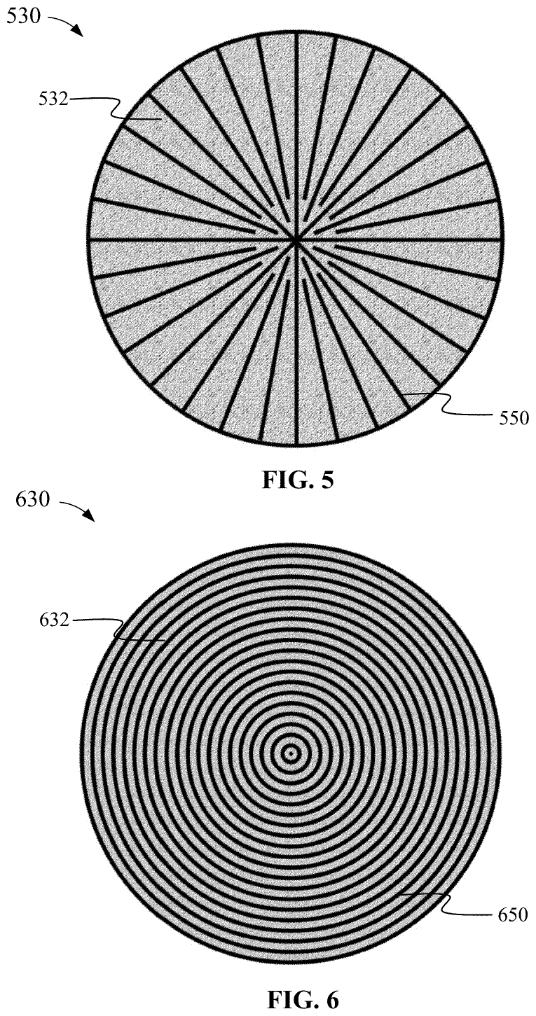

As described above with respect to the open architecture diffusion modules 230 and 330, channeled flow through the diffusion module may accelerate laminar flow in the radial direction, where localized flow paths may be progressively divided and expanded as flow is channeled through the diffusion module. In some variations, flow path expansion may be proportional to the length of the network of channels within the diffusion module. In some instances, the length of the network of interconnected channels within the diffusion module may be increased by increasing the dimension of the diffusion module in the direction of flow (e.g., the thickness of the module). The diffusion module 430 having a predetermined thickness as shown in FIG. 4(A) (e.g., taken along the cross-section A:A of the diffusion module 330 shown in FIG. 3) may be configured for even distribution of laminar flow in the radial direction. In some instances, adequate expansion of laminar flow in the radial direction may require an increased length of interconnected channels. In these instances, a diffusion module with thickness greater than that of the module described with reference to FIG. 4(A) may be used. For example, FIG. 4(B) shows a diffusion module that has a thickness about two times larger than the thickness of the diffusion module of FIG. 4(A). Additionally or alternatively, a plurality of diffusion modules of different thicknesses may be arranged in a serial configuration (e.g., stacking diffusion modules) in order to provide a predetermined length of interconnected channels.

In some variations, the diffusion module may have an axial thickness of between about 100 μm to about 200 μm, about 200 μm to about 400 μm, about 400 μm to about 800 μm, about 800 μm to about 1600 μm, about 1 mm to about 3 mm, about 3 mm to about 6 mm, about 6 mm to about 10 mm, about 1 cm to about 2 cm, about 2 cm to about 5 cm, and about 5 cm to about 15 cm, including all ranges and sub-values in-between.

In some variations, increasing the thickness of an open architecture diffusion module 330 may increase backpressure in fluids upon entering the diffusion module 330, thereby directing the radial spread of fluid across an external surface of the diffusion module 330. In some variations, increased laminar flow distribution towards a radial perimeter of the diffusion module 330 may correspond with decreasing the thickness of the diffusion module 330 at regions approaching its perimeter. In some variations, a thickness geometry of the diffusion module 330 may correspond to the outcomes targeted by the specific application in conjunction with associated flow requirements. For example, a predetermined laminar flow distribution may be achieved by linearly decreasing the thickness of the open architecture diffusion module 330 from the center towards the perimeter, as shown in FIG. 4(C). The slope of the surface of the diffusion module shown in FIG. 4(C) may be adjusted to a predetermined value in order to achieve other targeted outcomes for the laminar distribution of flow. In some variations, the thickness of the open architecture diffusion module 330 may be configured in various geometries in order to achieve the desired thickness along the radial direction. For example, in some variations, the thickness of the open architecture diffusion module 330 may assume a convex geometry as shown in FIG. 4(D). In some variations, the thickness of the open architecture diffusion module 330 may comprise a concave geometry, as shown in FIG. 4 (E). The examples of thickness geometries for the diffusion module 330 shown in FIGS. 4(A)-4(D) are presented for purposes of illustration and description and thus the systems and methods and/or concepts described herein are not intended to be limited to such specific examples.

In some variations, a diffusion module may comprise one or more non-porous inlays (e.g., on one or more surfaces), as illustrated in FIGS. 5-7. FIG. 5 shows an open architecture diffusion module 530 that includes non-porous inlays 550 on the surface 532 (e.g., the surface that faces the upstream inlet). The non-porous inlays 550 may be configured to distribute flow across an area proportional to the area occluded by the inlays 550. The non-porous inlays 550 on the surface 532 may occlude portions of the porosity of the diffusion module, thereby shunting flow to adjacent surfaces that have open porosity to facilitate permeation. As a result, fluid may be efficiently distributed across a predetermined region of the diffusion module 530, to expedite permeation. For example, fluid medium directed from the fluidic focal point of the diffusion module 530 may be efficiently distributed by one or more non-porous inlays 550 across the entire surface 532, allowing to achieve the desired outcome for the laminar distribution of flow. The non-porous inlays 550 may assume any suitable pattern in order to conform to the intended outcome for the laminar distribution of flow. For example, the non-porous inlays 550 shown in FIG. 5 comprise a radial patterning configured to shunt fluid towards an outer perimeter of the diffusion module 530 where flow permeates by radial diffusion into the porous portions (e.g., open porosity) unobstructed by the inlay. The radial patterning may be radially symmetrical, as shown in FIG. 5.

FIG. 6 shows an open architecture diffusion module 630 that includes non-porous inlays 650 on the surface 632 (e.g., the surface that faces the upstream inlet). In some variations, the non-porous inlays 650 have a concentric patterning configured to shunt fluid from the inlay proximal to the fluidic focal point of the diffusion module 630 towards the perimeter. In some variations, similar to the open architecture diffusion module 630 shown in FIG. 6, an open architecture diffusion module may include a spiral non-porous inlay approximating the concentric pattern shown in FIG. 6.

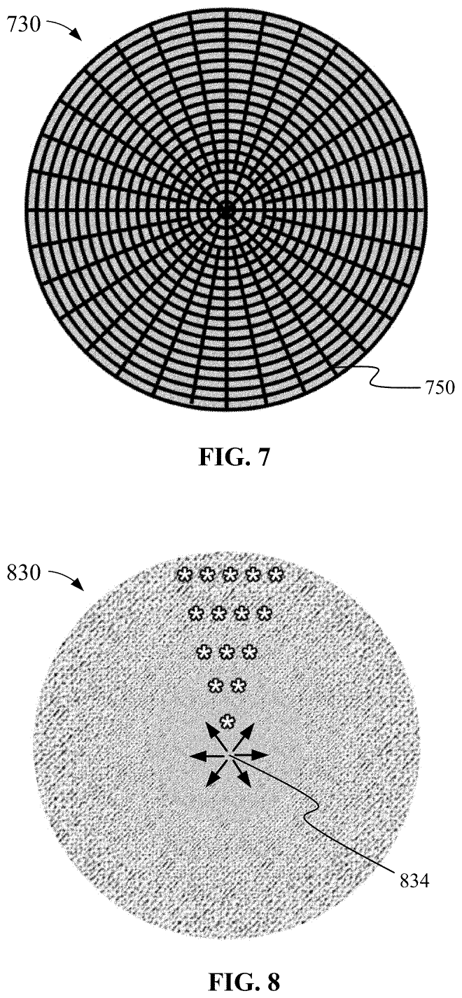

Aspects of radial and/or circular inlay patterning may be combined. For example, concentric and radial patterns may be combined, as shown in FIG. 7, to shunt fluid radially towards the perimeter of the diffusion module 730 in a generally even manner. The examples of patterns representing angular and radial directions shown in FIGS. 5-7 represent selected examples presented for purposes of illustration and description and thus the systems and methods and/or concepts described herein are not intended to be limited to such specific examples. In some variations, the thickness, length, shape, density, and distribution of the non-porous inlays is not limited by the figures described herein. For example, a density of a non-porous inlay pattern may decrease radially from a center to a perimeter of a diffusion module.

In some variations, the porosity and mean pore size of the diffusion module may additionally or alternatively be varied along the radial direction to achieve specific and/or desired outcomes for the laminar distribution of flow. For example, FIG. 8 shows an open architecture diffusion module 830 in which the porosity and/or mean pore size increases radially, as schematically represented by a larger number of stars per unit of area towards the perimeter of the diffusion module 830. Decreasing the porosity and/or the mean pore size of the diffusion module 830 may correspond to an increase in backpressure on the fluid located above the diffusion module, where fluid flows onto the module. By gradually increasing the porosity and/or the mean pore size from the fluidic focal point 834 to the perimeter of the diffusion module 830, localized accumulation of backpressure at the regions of lower porosity and/or smaller mean pore size may direct flow radially towards perimeter of the diffusion module, as represented in FIG. 8 by the arrows pointing towards the perimeter of the diffusion module 830. In some variations, the increased porosity and/or mean pore size may be linearly related to the radial distance from the fluidic focal point of the fluid.

In some variations, diffusion modules comprising an open architecture, such as those described in detail above with reference to the diffusion module 230, 330, 430, 530, 630, 730, and 830, may be less amenable to scalable applications involving high flow and high volume, given the increased structural constraints associated with fabricating diffusion modules of large radial dimensions. For these and any other suitable applications, enclosed architecture diffusion modules may enable a more tenable means to distribute laminar flow within a downstream conduit and/or substrate. Enclosed architecture diffusion modules apply the concepts described above with reference to the variations 230, 330, 430, 530, 630, 730 and 830, by channeling laminar flow first through an enclosed fluidic circuit including a central cavity or lumen of at least one hollow porous structure, for distal distribution of flow from the upstream inlet, and then through permeation and localized distribution through the porous walls of the hollow structure. Parameters (e.g., porosity, tortuosity, wall thickness, etc.) of the porous material in an enclosed architecture diffusion module or system may be similar to that described above for porous material in an open architecture diffusion module.

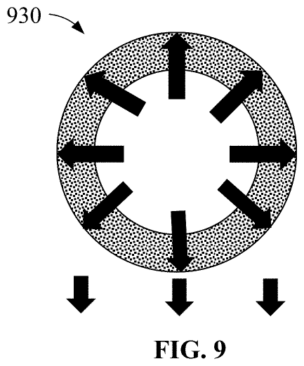

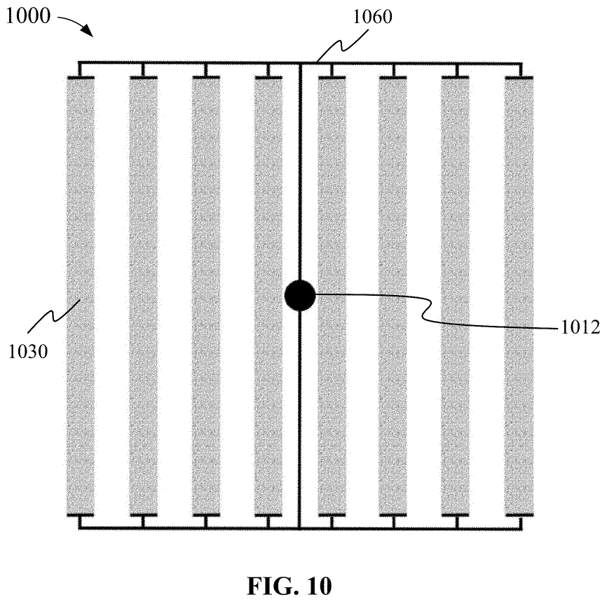

For example, FIG. 9 shows the cross-sectional view of a portion of an example variation of an enclosed architecture diffusion module 930. The diffusion module 930 may include a cylindrical member having a substantially circular radial cross-sectional shape (e.g., tubular structure) and a hollow interior forming a cavity or lumen. The walls of the diffusion module 930 may comprise a porous medium characterized by porosity, mean pore size and pore size distribution, where porosity refers to the percent void volume present within the material forming the walls of the diffusion module 930, as further described herein. The lumen inside the walls of the diffusion module 930 may be fluidically coupled to an upstream inlet (not shown) to receive fluid entering the inlet of the bioreactor. A fluid medium (e.g., inoculated with particles such as single cells, for example metazoan cells) may be transported in a laminar flow regime from the upstream inlet to a desired location where localized distribution through the porous walls surrounding the lumen may take place, as shown in FIG. 9 by the arrows oriented in the radial axis of the diffusion module 930. The tubular structure of the enclosed architecture diffusion module 930 may enable a disproportionately protracted ability to distribute flow in the radial axis direction (e.g., through the walls of the diffusion module 930) as compared to the axial direction (e.g., along the length of the tubular structure). Furthermore, as described below, a plurality of diffusion modules 930 may be connected with a manifold. Individual diffusion modules within the manifold may be conjoined at their ends and may be fluidically coupled to an upstream inlet. Although the enclosed architecture diffusion module 930 is shown in FIG. 9 as including a porous material around its entire cross-sectional area, it should be understood that in some variations, only a suitable portion of the cross-sectional area of the diffusion module may include a porous material (e.g., only a lower third, only a lower half, etc.).

FIG. 10 shows a top view of an enclosed architecture diffusion system 1000 according to another variation. The diffusion system 1000 may include an upstream inlet 1012, a plurality of individual diffusion modules 1030, and a conduit (e.g., manifold) 1060. The conduit may include a non-porous or suitable porous material in suitable connectors, etc. The upstream inlet 1012 may be located at any suitable position with respect to the diffusion modules 1030 and the manifold 1060. For example, as shown in FIG. 10, the upstream inlet 1012 may be located at a central position with respect to the diffusion modules 1030 and the manifold 1060, at a predetermined distance along the radial axis of said diffusion modules 1030. The upstream inlet 1012 may be fluidically coupled to the manifold 1060 and may be configured to flow fluid medium (e.g., in a laminar regime) in order to distribute flow to the individual diffusion modules 1030. The manifold 1060 may connect the individual diffusion modules 1030 according to any suitable configuration and/or geometrical arrangement. For example, the manifold 1060 may connect the individual diffusion modules 1030 in a parallel array, as shown in FIG. 10. The manifold 1060 may conjoin the ends of each individual diffusion module 1030, enabling distribution of laminar flow from the manifold into the lumen within the individual diffusion modules 1030. The distance from the upstream inlet 1012 to the ends of each diffusion modules 1030 may be based on a relative position of the diffusion module in the parallel array. For example, the distance from the upstream inlet 1012 to a first diffusion module located near the center of the parallel array of FIG. 10 may be smaller than the distance from the upstream inlet 1012 to a second diffusion module located at one of the ends of the parallel array.

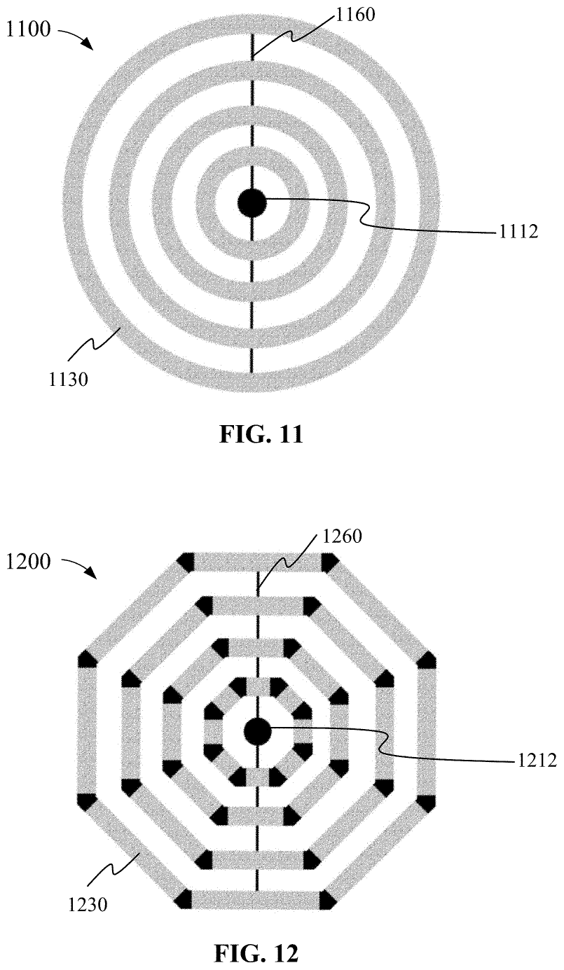

FIG. 11 shows a plan view of an enclosed architecture diffusion system 1100 according to another variation. The diffusion system 1100 may be similar to and/or substantially the same as the diffusion system 1000 described above with reference to FIG. 10, except the diffusion system 110 is shown with circular diffusion modules arranged in a concentric wheel or encircling arrangement. The diffusion system 1100 includes an upstream inlet 1112, a plurality of individual diffusion modules 1130, and a non-porous conduit 1160 (e.g., manifold). While each diffusion module 1130 is shown in FIG. 11 as having a circular shape, it should be understood that in some variations, one or more diffusion modules 1130 may have an elliptical or other suitable shape. The upstream inlet 1112 may be located at any suitable position with respect to the diffusion modules 1130 and the manifold 1160. For example, as shown in FIG. 11, the upstream inlet 1112 may be located at a central position with respect to the diffusion modules 1130 and the manifold 1160. The upstream inlet 1112 may be fluidically coupled to the manifold 1160 and may be configured to distribute flow to the individual diffusion modules 1130 for a laminar regime. The manifold 1160 may connect the individual diffusion modules 1130 in a concentric array, as shown in FIG. 11. In some variations, the manifold 1160 may couple individual diffusion modules 1130 in a coaxial geometry using a non-porous conduit extending in a radial direction. The distance from the upstream inlet 1112 to each of the diffusion modules 1130 may be based on the positions of the individual diffusion modules within the concentric array. For example, the distance from the upstream inlet 1112 to a first diffusion module located near the center of the concentric array of FIG. 11 is smaller than the distance from the upstream inlet 1112 to a second diffusion module located towards the outer regions of the concentric array.

FIG. 12 shows a top view of an enclosed architecture diffusion system 1200 according to another variation. The diffusion system 1200 may be similar to and/or substantially the same as the diffusion systems described above with reference to FIGS. 10 and 11, except the diffusion system 1200 is shown with polygonal diffusion modules arranged in a concentric wheel or encircling arrangement. The diffusion system 1200 includes an upstream inlet 1212, a plurality of individual diffusion modules 1230, and a non-porous conduit 1260 (e.g., manifold). Each diffusion module 1230 may include a polygon, where each side or segment of the polygon includes porous material such as that described above. Each side or segment of the polygon may be joined with a non-porous material (or additionally or alternatively a porous material) in suitable connectors, etc. Although each diffusion module 1230 is shown in FIG. 12 as having an octagonal shape, it should be understood that in some variations, one or more diffusion modules 1230 may have any suitable shape (e.g., triangular, square or rectangular, pentagonal, hexagonal, etc.). Furthermore, one or more polygonal diffusion modules 1130 may be combined with one or more circular or elliptical diffusion modules similar to diffusion modules 1030 described above with respect to FIG. 11. The upstream inlet 1212 may be fluidically coupled to the manifold 1260 and may be configured to flow fluid medium in laminar regime in order to distribute flow to the individual diffusion modules 1230. The manifold 1260 may connect the individual diffusion modules 1230 in a polygonal array, as shown in FIG. 12. The manifold 1260 may connect individual diffusion modules 1230 to the inlet port. The distance from the upstream inlet 1212 to each of the diffusion modules 1230 may be based on the positions of the individual diffusion modules within the polygonal array. For example, a first distance from the upstream inlet 1212 to a first diffusion module located near the center of the concentric array of FIG. 12 is smaller than a second distance from the upstream inlet 1212 to a second diffusion module located towards the outer regions of the polygonal array.

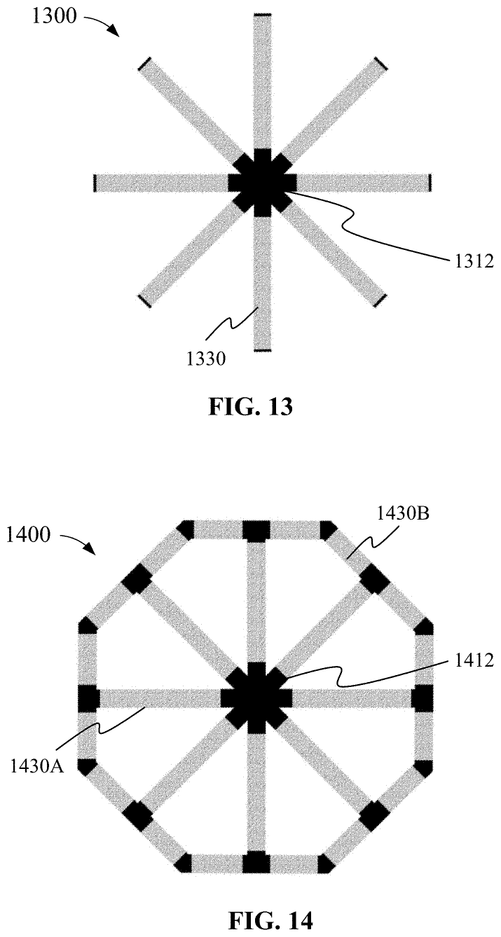

FIG. 13 shows a top view of an enclosed architecture diffusion system 1300 according to another variation. The diffusion system 1300 may be similar to and/or substantially the same as the diffusion systems described above with reference to FIGS. 10-12, except the diffusion system 1300 is shown with radially extending diffusion modules. The diffusion system 1300 may include an upstream inlet 1312 and a plurality of individual diffusion modules 1330. The individual diffusion modules 1330 may be organized in a radial array of “spokes” radially-extending from an upstream inlet 1312 arranged at a central hub, as shown in FIG. 13. Although the diffusion system 1300 is shown in FIG. 13 as including eight radially-extending diffusion modules 1330, it should be understood that in some variations the diffusion system may include any suitable number of radially-extending diffusion modules 1330 (e.g., two, three, four, five, six, seven, eight, nine, ten or more, etc.). Furthermore, the radially-extending diffusion modules 1330 may be equally or unequally radially distributed around the upstream inlet 1312.

FIG. 14 shows a top view of an enclosed architecture diffusion system 1400 according to another variation. The diffusion system 1400 may be similar to and/or substantially the same as the diffusion systems described above with reference to FIGS. 10-13, except the diffusion system 1400 is shown with combined features of systems 1100, 1200, and 1300 described above with reference to FIGS. 11, 12, and 13. The diffusion system 1400 may include an upstream inlet 1412, and a plurality of individual diffusion modules 1430A and 1430B organized according to a spoke and wheel array model. The individual diffusion modules 1430A may be organized in a radial array in which the ends of the one or more radially-extending diffusion modules 1430 proximal to the center of the radial array may be conjoined to the upstream inlet 1412, and the distal ends of the diffusion modules 1430 may be joined to one or more encircling diffusion modules 1430B as shown in FIG. 14. Other variations similar to the diffusion system 1400 may have any suitable number of radially-extending diffusion modules and/or any suitable number of encircling diffusion modules. Furthermore, each encircling diffusion module may have any suitable shape (e.g., circular, elliptical, polygonal) similar to that described above with respect to FIGS. 11 and 12.

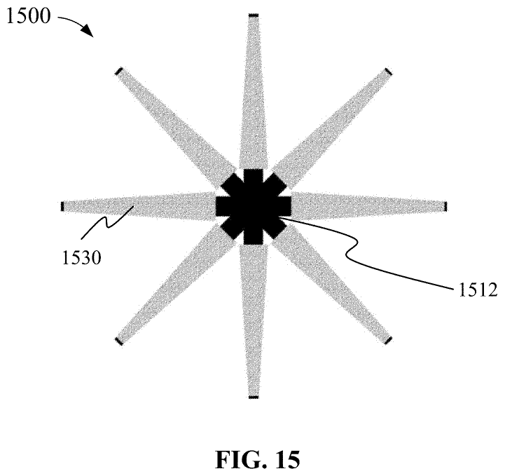

FIG. 15 shows a top view of an enclosed architecture diffusion system 1500 according to another variation. The diffusion system 1500 may be similar to and/or substantially the same as the diffusion system 1300 described above with reference to FIG. 13 with radially-extending diffusion modules, except that the diffusion system 1500 may include one or more diffusion modules with varying diameter and/or wall thickness along their length. The diffusion system 1500 may include an upstream inlet 1512, and a plurality of individual diffusion modules 1530. The individual diffusion modules 1530 may be organized in a radial array in which the upstream inlet 1512 is located at a position equidistant from every individual diffusion module 1530, as shown in FIG. 15. The individual diffusion modules 1530 may incorporate any of the concepts previously described with reference to open architecture diffusion modules such as variable thickness of the porous medium, addition of non-porous inlays, and/or porosity and pore size gradients along the length of the individual diffusion modules 1530. For example, FIG. 15 shows diffusion modules 1530 where the lumen wall of the diffusion modules 1530 decreases in thickness toward the radial perimeter of the radial array. In some variations, a relatively thicker porous medium wall comprising diffusion module 1530 may facilitate greater protraction of diffusion proximal to inlet 1512 where the radial density of the diffusion module 1530 is greatest. Conversely, a relatively thinner porous medium wall comprising diffusion module 1530 may facilitate reduced protraction of diffusion through the wall distal to the inlet 1512 where the radial density of diffusion module 1530 is least.

The examples of diffusion systems shown in FIGS. 10-15 are presented for purposes of illustration and description and thus the systems and methods and/or concepts described herein are not intended to be limited to such specific examples.

### II. Methods for Cultivating Tissue

Generally, a method for cultivating tissue may comprise directing a fluid comprising metazoan cells toward a diffusion module. The diffusion module may comprise a porous material that has at least one tortuous conduit extending between a first surface of the porous material and a second surface of the porous material. Fluid may flow through the porous material of the diffusion module, thereby radially expanding a flow of the fluid and seeding the metazoan cells onto a substrate proximate to the diffusion module. The method may incorporate use of one or more diffusion modules such as that described herein.

In some variations, a method for cultivating tissue may comprise directing a fluid comprising cells within a liquid medium toward one or more diffusion modules, with the first diffusion module comprising a first porous material that has at least one tortuous conduit extending between a first surface of the first porous material and a second surface of the first porous material and with the second diffusion module comprising a second porous material that has at least one tortuous conduit extending between a first surface of the second porous material and a second surface of the second porous material, passing the fluid through the first and the second porous material of the first diffusion module and the second diffusion module, thereby radially expanding a flow of the fluid; and seeding the metazoan cells onto a first side of a substrate and a second side of the substrate proximate to the first and second diffusion modules. The method may incorporate use of one or more diffusion modules such as those described herein.

For example, in some variations the method may be applied for cultivating tissue, such as cell-based meat products. The cell-based meat products of the disclosure are produced by the in vitro culturing of naturally occurring, genetically engineered, or modified animal cells in culture.

The methods provided herein are applicable to any metazoan cell in culture. Generally, the cells are from any metazoan species whose tissues are suitable for dietary consumption. In some variations the cells may demonstrate a capacity for differentiation into mature tissue, such as skeletal muscle tissue, other muscle tissues, or any cell, cellular biomass, and/or tissue that can be consumed as meat. The cells of the present disclosure may be primary cells, or cell lines. The cells may be adherent-cells or non-adherent cells.

In some variations, the cells are derived from any non-human animal species intended for human or non-human dietary consumption (e.g. cells of avian, ovine, caprine, porcine, bovine, piscine origin) (e.g. cells of livestock, poultry, avian, game, or aquatic species, etc.).

In some variations, the cells are from livestock such as domestic cattle, pigs, sheep, goats, camels, water buffalo, rabbits and the like. In some variations, the cells are from poultry such as domestic chickens, turkeys, ducks, geese, pigeons and the like. In some variations, the cells are from game species such as wild deer, gallinaceous fowl, waterfowl, hare and the like. In some variations, the cells are from aquatic species or semi-aquatic species harvested commercially from wild fisheries or aquaculture operations, or for sport, including certain fish, crustaceans, mollusks, cephalopods, cetaceans, crocodilians, turtles, frogs and the like.

In some variations, the cells are from exotic, conserved or extinct animal species. In some variations, the cells are from Gallus gallus, Gallus domesticus, Bos taurus, Sous scrofa, Meleagris gallopavo, Anas platyrynchos, Salmo salar, Thunnus thynnus, Ovis aries, Coturnix, Capra aegagrus hircus, or Homarus americanus. Accordingly, exemplary cell-based meat products of the disclosure include avian meat products, chicken meat products, duck meat products, and bovine meat products.

In some variations, the cells are primary stem cells, self-renewing stem cells, embryonic stem cells, pluripotent stem cells, induced pluripotent stem cells, or transdifferentiated pluripotent stem cells.

In some variations, the cells are modifiable by a genetic switch to induce rapid and efficient conversion of the cells to skeletal muscle tissue, connective tissue, fat tissue, and/or any other mature tissue for cultured meat production.

In some variations, the cells are myogenic cells, destined to become muscle, or muscle-like cells. In some variations, the myogenic cells are natively myogenic, e.g. myoblasts. Natively myogenic cells include, but are not limited to, myoblasts, myocytes, satellite cells, side population cells, muscle derived stem cells, mesenchymal stem cells, myogenic pericytes, or mesoangioblasts.

In some variations, cells are of the skeletal muscle lineage. Cells of the skeletal muscle lineage include myoblasts, myocytes, and skeletal muscle progenitor cells, also called myogenic progenitors that include satellite cells, side population cells, muscle derived stem cells, mesenchymal stem cells, myogenic pericytes, and mesoangioblasts.

In other variations, the cells are not natively myogenic (e.g. are non-myogenic cells such as fibroblasts or non-myogenic stem cells that are cultured to become myogenic cells in the cultivation infrastructure).

In some variations, the cells of the cellular biomass are somatic cells. In some variations, the cells of the cellular biomass are not somatic cells.

In some variations the cells are genetically edited, modified, or adapted to grow without the need of specific ingredients including specific amino acids, carbohydrates, vitamins, inorganic salts, trace metals, TCA cycle intermediates, lipids, fatty acids, supplementary compounds, growth factors, adhesion proteins and recombinant proteins.

In some variations, the cells may comprise any combinations of the modifications described herein.

The cell-based meat of the present disclosure, generated using the cell media formulations provided herein, is suitable for both human and non-human consumption. In some variations, the cell-based meat is suitable for consumption by animals, such as domesticated animals. Accordingly, the cell media formulations provided herein support the growth of “pet food”, e.g. dog food, cat food, and the like.

In some variations the methods described herein may enable production of thick tissues without the need for an internal scaffold to support tissue dimensionality.

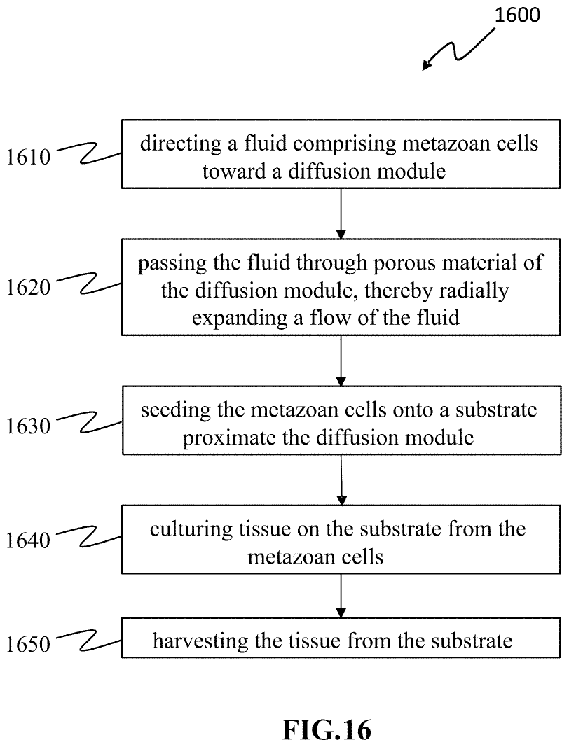

FIG. 16 is a flowchart that generally describes a method (1600) using any of the systems and devices described herein. The method (1600) may include directing a fluid (e.g., fluid medium, cell culture medium, etc.) toward a diffusion module. In some variations, the fluid may comprise any of the cell types described herein (e.g., metazoan cells) and/or a liquid such as a liquid nutrient medium. The diffusion module may comprise a porous material having at least one tortuous conduit extending between a first surface of the porous material and a second surface of the porous material. In some variations, the fluid may be directed at any predetermined angle and flow rate. For example, the fluid may be directed in a perpendicular or non-perpendicular direction relative to a surface of the diffusion module. The fluid may be directed from one or more directions towards one or more portions of the diffusion module. In some variations, a diameter of the fluid flow may be less than a diameter of the diffusion module. For example, an inlet through which the fluid is directed towards the diffusion module may have a radius less than a radius of the diffusion module. In some variations, the ratio between a radius of the upstream inlet to a radius of the housing may be any of the ranges described in more detail herein. The method may incorporate use of one or more diffusion modules such as that described herein.

In some variations, the fluid may be passed through the porous material of the diffusion module thereby facilitating radially expanding laminar flow of the fluid (1620) by transitioning turbulent flow of the fluid into laminar flow. In some variations, radially expanding a flow of the fluid may comprise radially distributing the cells across the diffusion module. In some variations, the radial expansion of laminar flow due to the diffusion module may be increased by any of the ranges described herein.

In some variations, the fluid passed through the diffusion module may be directed towards a substrate proximate the diffusion module. In some variations, the cells (e.g., metazoan cells) may be seeded onto the substrate proximate the diffusion module (1630). The seeded cells may be anchored to the substrate (e.g., a surface of the substrate). Thereafter, seeded (e.g., anchored) cells may receive fluid through the porous material. In some variations, fluid may be directed towards opposite sides of a substrate in an alternating manner. For example, FIG. 2C depicts a first diffusion module 230A configured to seed a first side (e.g., top side) of the substrate 220, and a second diffusion module 230B configured to seed a second side (e.g., bottom) of the substrate 220. The first side may be opposite the second side. In FIG. 2C, a relative angle of the alternating flow trajectories may be about 180 degrees, but may be any of the angles described herein.

After exposure to the fluid medium for a first time period, the flow of the bioreactor may be reversed under a second flow for a second time period. The fluid flow may be alternated between the first flow and the second flow a predetermined number of times, as described in more detail herein. During reversed flow, fluid medium may flow from the downstream outlet, through the diffusion module onto 230B and over the downstream facing side of the substrate, as shown in the right side bioreactor of FIG. 2C.

In some variations, tissue may be cultured on the substrate from the cells (e.g., metazoan cells) (1640). The diffusion module may be configured to reduce variation in the hydrodynamic shear force of the fluid exerted upon the seeded cells, thereby improving one or more of distribution, adhesion, growth, and support of the cell culture. For example, the diffusion module may enable a more even density of seeded cells across a surface of a substrate and allow control of fluid flow and shear forces across a larger surface area of a substrate. This may allow the seeded cells to receive nutrients and grow in a more consistent manner, thereby increasing tissue harvest yields.

In some variations, the tissue may be harvested from the substrate (1650). In some variations, tissue may be separated (e.g., harvested) from the substrate by one or more mechanisms including enzymatic, chemical, and mechanical processes. In some variations, the substrate may be configured to degrade (e.g., resorb). For example, tissue harvesting may comprise one or more of fluidic, spontaneous, chemical, electrical, optical, thermal, and mechanical detachment. For example, one or more buffers or enzymatic solutions contact the cells to induce detachment from the substrate. In some embodiments, one or more of a volume and rate of fluid flow may be increased to harvest tissue. The tissue may be separated from the substrate and then collected within or outside the bioreactor.

The foregoing description, for purposes of explanation, used specific nomenclature to provide a thorough understanding of the invention. However, it will be apparent to one skilled in the art that specific details are not required in order to practice the invention. Thus, the foregoing descriptions of specific variations of the invention are presented for purposes of illustration and description. They are not intended to be exhaustive or to limit the invention to the precise forms disclosed; obviously, many modifications and variations are possible in view of the above teachings. The variations were chosen and described in order to explain the principles of the invention and its practical applications, they thereby enable others skilled in the art to utilize the invention and various variations with various modifications as are suited to the particular use contemplated. It is intended that the following claims and their equivalents define the scope of the invention.

1. A system for cultivating tissue, the system comprising:
a bioreactor comprising an inlet;
a substrate arranged in the bioreactor; and
a diffusion module configured to transfer fluid from the inlet to the substrate, wherein the diffusion module comprises a porous material having at least one tortuous conduit extending between a first surface of the porous material and a second surface of the porous material.

2. The system of claim 1, wherein the at least one tortuous conduit has a mean pore size of between about 5 nm and about 10 nm, between about 10 nm and about 20 nm, between about 20 nm and about 40 nm, between about 40 nm and about 80 nm, between about 80 nm and about 160 nm, between about 160 nm and about 320 nm, between about 320 nm and about 640 nm, between about 0.64 μm and about 1.2 μm, between about 1.2 μm and about 2.4 μm, between about 2.4 μm and about 4.8 μm, between about 4.8 μm and about 10 μm, between about 10 μm and about 20 μm, between about 20 μm and about 40 μm, between about 40 μm and about 80 μm, between about 80 μm and about 160 μm, between about 160 μm and about 320 μm, between about 320 μm and about 600 μm, or between about 0.6000 mm and about 1.2 mm.

3. The system of claim 1, wherein the at least one tortuous conduit has an average arc to chord length ratio of between about 1.1 to about 1.2, about 1.2 to about 1.4, about 1.4 to about 1.6, about 1.6 to about 1.8, about 1.8 to about 2.0, about 2 to about 3, about 3 to about 4, about 4 to about 5, about 5 to about 6, about 6 to about 7, about 7 to about 8, about 8 to about 9, about 9 to about 10, about 10 to about 12, about 12 to about 14, about 14 to about 16, about 16 to about 18, about 18 to about 20, about 20 to about 25, about 25 to about 30, about 30 to about 35, about 35 to about 40, about 40 to about 45, about 45 to about 50, about 50 to about 60, about 60 to about 70, about 70 to about 80, about 80 to about 90, or about 90 to about 100.

4. The system of claim 1, wherein a porosity of the porous material is between about 0.1% to about 0.25%, about 0.25% to about 0.50%, about 0.50% to about 1.0%, about 1.0% to about 2.5%, about 2.5% to about 5.0%, about 5.0% to about 10%, about 10% to about 20%, about 20% to about 30%, about 30% to about 40%, about 40% to about 50%, about 50% to about 60% about 60% to about 70% about 70% to about 80%, about 80% to about 90%, about 90% to about 95%, or about 95% to about 99%.

5. The system of claim 1, wherein the diffusion module comprises a fluidic focal point aligned with the inlet.

6. The system of claim 5, wherein the diffusion module has at least one geometrical characteristic that varies with distance from the fluidic focal point.

7. The system of claim 6, wherein the at least one geometrical characteristic comprises porosity that increases with distance from the fluidic focal point.

8. The system of claim 1, wherein the diffusion module comprises a planar surface.

9. The system of claim 1, wherein the diffusion module comprises a concave surface.

10. The system of claim 1, wherein the diffusion module comprises a convex surface.

11. The system of claim 1, wherein the diffusion module comprises an open surface in fluidic communication with the inlet.

12. The system of claim 1, wherein the diffusion module is one of a plurality of diffusion modules each comprising a fluidic channel comprising the porous material, wherein the system further comprises a fluidic circuit comprising a manifold in fluidic communication with the inlet and the fluidic channels.

13. The system of claim 12, wherein at least a portion of the fluidic channels are arranged in parallel.

14. The system of claim 12, wherein at least a portion of the fluidic channels extend radially from a fluidic focal point of the diffusion module.

15. The system of claim 12, wherein at least a portion of the fluidic channels are arranged in concentric circuits around a fluidic focal point of the diffusion module.

16. The system of claim 1, wherein the diffusion module comprises a non-porous material arranged adjacent the porous material.

17. The system of claim 16, wherein the non-porous material is inlaid in the porous material.

18. The system of claim 16, wherein the surface area ratio of the non-porous material to the porous material decreases with distance from a fluidic focal point of the diffusion module.

19. The system of claim 1, wherein the porous material comprises at least one material selected from the group consisting of: a silicate, a ceramic, a carbon allotrope, a metal, metallic alloy, a synthetic polymer, a biological polymer, a synthetically-modified biological polymer, a composite, and a resin.

20. The system of claim 1, further comprising at least another diffusion module, wherein the at least another diffusion module is between a second inlet of the bioreactor and the substrate.

21.-39. (canceled)
